(19) United States
(12) Patent Application Publication
(10) Pub. No.: US 2012/0021036 A1
(43) Pub. Date: Jan. 26, 2012

(54) COMPOSITE NANOSTRUCTURES AND METHODS FOR MAKING AND USING THEM

(75) Inventors: Bharat Majeti, La Jolla, CA (US); Eric Murphy, San Marcos, CA (US); Wolfgang Wrasidlo, La Jolla, CA (US); David Cheresh, Encinitas, CA (US)

(73) Assignee: THE REGENTS OF THE UNIVERSITY OF CALIFORNIA, Oakland, CA (US)

(21) Appl. No.: 13/143,825

(22) PCT Filed: Jan. 14, 2010
(86) PCT No.: PCT/US2010/021077

§ 371 Date: Sep. 29, 2011

Related U.S. Application Data
(60) Provisional application No. 61/145,071, filed on Jan. 15, 2009.
(60) Provisional application No. 61/165,425, filed on Mar. 31, 2009.

(51) Int. Cl. A61K 9/14; A61K 31/7068; A61K 38/22; A61K 31/196; A61K 31/675; A61K 31/519; A61K 31/52; A61K 31/513; A61K 31/704; A61K 38/14; A61K 31/135; A61K 31/167; A61K 31/573; A61K 38/46; A61K 33/24; A61K 31/7048; A61K 38/19; A61K 31/337; A61K 38/20; A61K 31/437; A61P 35/00; A61K 9/00; B82Y 5/00

(52) U.S. Cl. 424/422; 424/400; 424/498; 514/49; 514/9.7; 514/19.3; 514/567; 514/110; 514/249; 514/263.3; 514/274; 514/34; 514/2.3; 514/653; 514/629; 514/180; 424/94.6; 424/85.4; 424/85.5; 424/85.6; 424/85.7; 424/649; 514/27; 424/85.1; 514/449; 424/85.2; 514/285; 977/773

(57) ABSTRACT

The invention provides nanostructures or products of manufacture for use as ex vivo or in vivo composition (e.g., a drug, or a therapeutic, diagnostic or imaging reagent) delivery vehicles. In one aspect, the invention provides nanoparticles comprising several compartments which in unison function as a composite nanostructure. In one embodiment, the nanoparticles of the invention comprise a combination of polymer core/lipid bilayer interface which incorporate covalently attached lipid-vascular targeting ligands. These composite nanoparticles can deliver highly effective and selective payloads for diagnostic, prophylactic or therapeutic applications.

### FEDERAL FUNDING

This invention was made with Federal government support under by grants from the NCI Nanotechnology Alliance (DAC) and NIH: P01-CA078045 (DAC), P01-HL057900 (DAC), and P01-CA 104898 (DAC), National Institutes of Health. The Federal government has certain rights in this invention.

### FIELD OF THE INVENTION

The invention provides nanostructures or products of manufacture for use as ex vivo or in vivo composition (e.g., a drug, or a therapeutic, diagnostic or imaging reagent) delivery vehicles. In one aspect, the invention provides nanoparticles comprising several compartments which in unison function as a composite nanostructure. In one embodiment, the nanoparticles of the invention comprise a combination of polymer core/lipid bilayer interface which incorporate covalently attached lipid-vascular targeting ligands. These composite nanoparticles can deliver highly effective and selective payloads for diagnostic, prophylactic or therapeutic applications.

### BACKGROUND

Nanoparticles have been described for a variety of technical applications, including as drug delivery and release vehicles. Hollow nanospheres of about 50 to 200 nm diameter have been described. Some have pores in their shells to allow chemical dissolution of interior materials; this can allow a passive drug delivery based on slowed-down kinetics.

### SUMMARY

In alternative embodiments, the invention provides nanostructures or products of manufacture for use as an in vivo, ex vivo or in vitro (in culture) delivery vehicle for a composition, e.g. a drug or a diagnostic, prophylactic or therapeutic reagent, or to deliver compositions for diagnostic, prophylactic or therapeutic applications; e.g., for the in vivo, ex vivo or in vitro (in culture) delivery of a therapeutic, diagnostic or imaging reagent. In alternative embodiments, the invention provides nanostructures or products of manufacture and methods for making and using them.

In alternative embodiments the invention provides products of manufacture or composite nanostructures for in vitro, in vivo and/or ex vivo composition delivery comprising:

(i) a polymer core, wherein the polymer comprises a plurality of monomers;

(ii) a first lipid or phospholipid layer or coating over (substantially covering) the polymer core;

(iii) a composition entirely contained within the polymer core, or composition substantially contained within the polymer core; and

(iv) a cell or tissue targeting composition covalently, ionically or non-covalently attached to the lipid or phospholipid such that a cell or tissue targeting of the cell or tissue targeting composition is positioned outward (to the exterior) of the product of manufacture or composite nanostructure.

In one embodiment of the product of manufacture or a composite nanostructure of (a), the nanoparticle formulation comprises (PEG is polyethylene glycol): cholesterol:DOPE:DSPC:DSPE-(PEO)₄-cRGDfK:DSPE-mPEG2000 at a 6:6:6:1:1 molar ratio.

In alternative embodiments of the products of manufacture or composite nanostructures of the invention, wherein the polymer core is processed or manufactured to comprise a size in a range of between about 2, 3, 4, 5, 6, 7, 8, 9, 10, 11, 12, 13, 14, 15, 16, 17, 18, 19 or 20 or more to about 10, 50, 100, 200, 300, 400, 500, 600, 700, 800, 900 or 1000 or more nm; or, the product of manufacture or a composite nanostructure is processed or manufactured to have a size range of from between about 2, 3, 4, 5, 6, 7, 8, 9, 10, 11, 12, 13, 14, 15, 16, 17, 18, 19 or 20 or more to about 10, 50, 100, 200, 300, 400, 500, 600, 700, 800, 900 or 1000 or more nm.

In alternative embodiments of the products of manufacture or composite nanostructures of the invention, the polymer core comprises or consists of one or more materials or compositions capable of absorbing hydrophobic molecules, hydrophilic molecules or hydrophobic molecules and hydrophilic molecules. The polymer core can comprise or consist of a material capable of forming a hydrogel, and/or the polymer core can comprise or consist of random and/or block copolymers. The polymer hydrogel core can comprise or consist of a methacrylic or acrylic ester monomer, an acrylamide (methacrylamide) monomer, an N-vinyl-2-pyrrolidone, a starch, ethylene glycol, hyaluran, chitose, and/or cellulose, or equivalents. The polymer core can comprise or consist of a polypeptide, peptide or peptidomimetic; and in alternative embodiments, the polypeptide, peptide or peptidomimetic comprises or consists of a biopolymer, e.g., comprises or consists of an albumin or equivalent.

In alternative embodiments of the products of manufacture or composite nanostructures of the invention, the monomers, the polymer hydrogel and/or the polypeptide, peptide or peptidomimetic are cross-linked with a cross-linking agent; or the core comprises or consists of an unsaturated moiety capable of ultraviolet (uv) or thermal crosslinking to stabilize the core material. The monomers, the polymer hydrogel and/or the polypeptide, peptide or peptidomimetic can be cross-linked with a cross-linking agent or cross-linking mechanism comprising or consisting of a radical mechanism or a chemical crosslinking agent. The chemical crosslinking agent can comprise or consist of a glutaraldehyde, or equivalent. The cross-linking agent can comprise or consist of ethylene dimethacrylate, N,N-methylenediacrylamide, methylenebis(4-phenyl isocyanate), epichlarohydin glutaraldehyde, ethylene dimethacrylate, divinylbenzene and/or allyl methacrylate, or equivalent. The polymer core can comprise or consist of a polylactide (PLA), a polyglycolide (PGA), a polylactide coglycolide (PLGA), a polycaprolactone (PCL), a copolymer of these materials, a polyanhydride, a polyortho ester, a biostable or bioinert hydrogel matrix-forming polymer, a water-containing gel, a polyvinylpyrrolidone (PVP), a polyethylene glycol (PEG), a polyethylene oxide (PEO), a polyacrylamide (PAA), polyvinyl alcohol (PVA), a biostable or bioinert matrix-forming polymer, a synthetic polymer, a methyl methacrylate, a butyl methacrylate and/or a dimethyl siloxane, or equivalent.

In alternative embodiments of the products of manufacture or composite nanostructures of the invention, the monomers, the cell or tissue targeting composition is covalently, ionically or non-covalently attached to the lipid or phospholipid via a cross-linking agent.

In alternative embodiments of the products of manufacture or composite nanostructures of the invention, the monomers, the cross-linking agent comprises or consists of ethylene dimethacrylate, N,N-methylenediacrylamide, methylenebis(4-phenyl isocyanate), epichlarohydin glutaraldehyde, ethylene dimethacrylate, divinylbenzene and/or allyl methacrylate, or equivalent.

In alternative embodiments of the products of manufacture or composite nanostructures of the invention, the monomers, the products of manufacture or composite nanostructures of the invention comprise an imaging agent, a therapeutic agent (e.g., a radionuclide), a drug, a biological agent, a small molecule, a chemical, a cell, or an inorganic nanoparticle; and in alternative aspects these compositions are included in the products of manufacture or composite nanostructures of the invention for targeted delivery in vivo, ex vivo or in vitro (in culture).

In alternative embodiments of the products of manufacture or composite nanostructures of the invention, the cell or tissue targeting composition comprises a ligand capable of specifically binding to an integrin, or equivalent. The integrin can comprise or consist of (or is) avb3, a4b1 a5b1 and/or plaktin, or equivalent.

In alternative embodiments of the products of manufacture or composite nanostructures of the invention, the cell or tissue targeting composition comprises or consists of (or is) a linear peptide, a cyclic peptide or a synthetic organic molecule mimicking a peptide. The cell or tissue targeting composition can comprise or consist of (or is) an antibody.

In alternative embodiments the products of manufacture or composite nanostructures of the invention further comprise a second lipid layer or coating. The second lipid layer or coating can act as a stealth component on a first lipid layer or can be a coating monolayer to improve biological compatibility and increase in vivo circulation rates. The first lipid layer or coating or the second lipid layer or coating can further comprise a stabilizing molecule. The stabilizing molecule can comprise or consist of (or is) a cholesterol or a colesteric analog, or comprises or consists of (or is) a crosslinkable lipid that is UV or heat sensitive.

In alternative embodiments of the products of manufacture or composite nanostructures of the invention, the product of manufacture or a composite nanostructure further comprise a component positioned (sandwiched) between the first and second lipid layer or coating to increase the melting range of the first and second lipid layer or coating monolayer component.

In alternative embodiments of the products of manufacture or composite nanostructures of the invention, the product of manufacture or a composite nanostructure further comprise a crosslinked lipid to stabilize the first and/or second lipid layer.

The first and/or second lipid or phospholipid layer or coating can comprise a free saturated or unsaturated fatty acid or an ester or an amide thereof; an anionic lipid, a cationic lipid, a zwitterionic lipid, a diacyl trialkylammonium propane and/or a quaternary ammonium compound, or equivalent. The first and/or second lipid or phospholipid layer or coating can comprise a mixture of oil-phase components, a squalane, a sterol, a ceramide, a neutral lipid or oil, a fatty acid, a lecithin, a phosphatidylcholine (PC) diacyl ester, a PC with saturated or unsaturated fatty acids or with aromatic acids such as dioleoyl-PC, dimyristoyl-PC (DMPC), dipalmitoyl-PC, distearoyl-PC (DSPC), diarachidonyl-PC (DAPC), and diphthaloyl-PC (DPPC); phosphatidylethanolamine (PE) diacyl esters with saturated or unsaturated fatty acids or with aromatic acids such as diolcoyl-PE, dimyristoyl-PE (DMPE), dipalmitoyl-PE (DPPE) and distearoyl-PE (DSPE); phosphatidylserine; phosphatidylglycerols, distearoylphosphatidylglycerol (DSPG); phosphatidylinositol; phosphatidic acids (PA), dipalmitoyl-PA (DPPA) and/or distearoyl-PA (DSPA), or equivalent. The first and/or second lipid or phospholipid layer or coating can comprise a lipid-bearing polymer such as chitin, hyaluronic acid, polyvinylpyrrolidone or polyethylene glycol (PEG), a pegylated DPPE (DPPE-PEG), phosphatidylcholine, phosphatidylethanolamine, sphingomyelin, phosphatidyl-glycerol, phosphatidylserine, phosphatidylinositols, phosphatidic acid, stearylamine, diacyloxy trimethylammonium propanes and/or diacyloxy dimethylammonium propane.

In alternative embodiments the products of manufacture or composite nanostructures of the invention comprise for targeted delivery (e.g., in vivo, ex vivo or in vitro (in culture)) a bioactive material, an anticancer cytostatic or cytotoxic agent, a gemcitabine (e.g., GEMZAR™) or a small molecule inhibitor of oncogenic or inflammatory proteins, a kinase inhibitor, a cytotoxic lipopeptide, a somocystinamide or a curacin A; or an antibiotic, an antidepressant, an anti-tumorigenic, an antiviral, a cytokine, a hormone, an imaging agent, a neurotransmitter, a nucleic acid, a stimulant, a regulating agent that turns genes and/or protein production on or off; a poly-functional alkylating agent, mechlorethamine, chlorambucil, melphalan, thiotepa, busulfan, cyclophosphamide, ifosfamide, an antimetabolite, methotrexate, 6-mercaptopumme, 6-thioguanme, 5-fluorouracil, 5-fluorodeoxyuridine, cytarabine, fludarabine, 2-chlorodeoxyadenosine, 2-deoxycoformycin, genicitabine, an antibiotic, doxorubicin, bleomycin, dactinomycin, daunorubicin, plicamycin, mitomycin C, mitoxantrone, a steroid, a hormonally active compound, androgen, fluoxymesterone, an anti-androgen, flutamide, an estrogen, ethinyl estradiol, diethylstilbestrol, an anti-estrogen, tamoxifen, a progestational agent, megestrol acetate, a luteinizing hormone-releasing hormone agonist, leuprolide, an aromatase inhibitor, aminoglutethimide, an adrenal cortical compound, dexamethasone, asparaginase, altretaimine, carmustine, lomustine, steptozotocin, mitotane, dacarbazine, hydroxyurea, etoposide, cisplatin, carboplatin, procarbazine, vinblastine, vincristine, levamisole, cis-retinoic acid, paclitaxel, docetaxel, a biologic agent, alpha-interferon, beta-interferon, interferon-gamma, tumor necrosis factor, erythropoietin, granulocyte colony-stimulating factor, granulocyte macrophage colony-stimulating factor, macrophage colony-stimulating factor or interleukin-1, interleukin-2 and combinations thereof.

In alternative embodiments the invention provides devices for treating or ameliorating a disease or condition comprising the product of manufacture or nanostructure of the invention, wherein optionally the product of manufacture or nanodevice comprises or is contained within an implant or an intradermal or a subcutaneously transplantable assembly.

In alternative embodiments the invention provides implants comprising the products of manufacture or nanostructures of the invention, wherein optionally the implant is an intradermal or a subcutaneously transplantable assembly.

In alternative embodiments the invention provides anti-nerve gas agent, or anti-toxin, devices comprising the product of manufacture or nanostructure of the invention, wherein optionally the product of manufacture or nanodevice comprises or is contained within an implant, or an intradermal or a subcutaneously transplantable assembly.

Biological agents that can be stored in (and delivered by) the nanostructures and products of manufacture of this invention include growth factors, collagens, various proteins/biomolecules, genes, enzymes, hormones, nucleic acids (e.g., DNA or RNA), antibiotics, drugs and functional nanoparticles. The nanostructures or products of manufacture of the invention can comprise any desired material, composition or agent, e.g., dyes, contrasting agents, drugs, growth factors, hormones, antibiotics, antibodies, nucleic acids, lipids, polypeptides (including enzymes, peptides, peptidomimetics), carbohydrates, other nanoparticles, nutrients, vitamins, and minerals.

The nanostructures can be used e.g., for drug treatments, for prophylactic reasons, for cell growth, e.g., stem cell growth, or enhancing hepatocyte growth, or stimulating vascularization and/or other cell growth and functionalities.

The nanostructures or products of manufacture of the invention can be administered in any manner, e.g., as subcutaneous or intradermal implants or in transplantations, including in implants comprising a three-dimensional array.

The nanostructures or products of manufacture of the invention can be administered to any cell type, e.g., compounds contained in nanostructures or products of manufacture of the invention can be for accelerating bone growth for orthopedic and dental repair; in vivo, ex vivo or in vitro (in culture) accelerated growth of cells including functional cells (such as liver cells, kidney cells, nerve cells, myocytes, stem cells) or supportive tissues (soft tissues such as muscles, tendons, fibrous tissues, periodontal tissues, fat, blood vessels, or hard tissues such as bone and teeth), proliferation and/or harvesting of cells to be supplied for therapeutics and laboratory experiments, particularly rare cell types such as stem cells or disease cells; therapeutic applications for local sustained drug release; and rapid diagnosis of cell-based conditions, toxicities and/or diseases involved in, for example, infections, epidemics and/or biological warfare agent or toxin exposures.

In alternative embodiments, the invention provides pharmaceutical compositions or formulations for treating or ameliorating a disease or condition comprising the product of manufacture of the invention, wherein optionally the product of manufacture or nanostructure comprises or is contained within an implant or an intradermal or a subcutaneously transplantable assembly. The pharmaceutical composition or formulation can be formulated for enteral or parenteral administration.

In alternative embodiments, the invention provides uses of the product of manufacture or nanostructure of the invention, to make a pharmaceutical composition or formulation, for e.g., treating or ameliorating a cancer, an autoimmune disease or an infection, wherein optionally the pharmaceutical composition or formulation comprises an antibiotic, an antidepressant, an anti-tumorigenic, an antiviral, a cytokine, a hormone, an imaging agent, a neurotransmitter, a nucleic acid, a stimulant, a regulating agent that turns genes and/or protein production on or off, a poly-functional alkylating agent, mechlorethamine, chlorambucil, melphalan, thiotepa, busulfan, cyclophosphamide, ifosfamide, an antimetabolite, methotrexate, 6-mercaptopumme, 6-thioguanme, 5-fluorouracil, 5-fluorodeoxyuridine, cytarabine, fludarabine, 2-chlorodeoxyadenosine, 2-deoxycoformycin, genicitabine, an antibiotic, doxorubicin, bleomycin, dactinomycin, daunorubicin, plicamycin, mitomycin C, mitoxantrone, a steroid, a hormonally active compound, androgen, fluoxymesterone, an anti-androgen, flutamide, an estrogen, ethinyl estradiol, diethylstilbestrol, an anti-estrogen, tamoxifen, a progestational agent, megestrol acetate, a luteinizing hormone-releasing hormone agonist, leuprolide, an aromatase inhibitor, aminoglutethimide, an adrenal cortical compound, dexamethasone, asparaginase, altretaimine, carmustine, lomustine, steptozotocin, mitotane, dacarbazine, hydroxyurea, etoposide, cisplatin, carboplatin, procarbazine, vinblastine, vincristine, levamisole, cis-retinoic acid, paclitaxel, docetaxel, a biologic agent, alpha-interferon, beta-interferon, interferon-gamma, tumor necrosis factor, erythropoietin, granulocyte colony-stimulating factor, granulocyte macrophage colony-stimulating factor, macrophage colony-stimulating factor or interleukin-1, interleukin-2 and combinations thereof.

All publications, patents, patent applications, GenBank sequences and ATCC deposits, cited herein are hereby expressly incorporated by reference for all purposes.

### BRIEF DESCRIPTION OF THE DRAWINGS

FIG. 1 illustrates a schematic of an exemplary method for preparing exemplary nanogels of the invention using the exemplary method comprising extrusion and UV crosslinking with a photoinitiator, as discussed in detail in Example 1, below.

FIG. 2 graphically illustrates data from a cell viability assay comparing an exemplary integrin αvβ3 targeted nanogel loaded with various small molecules, as discussed in detail in Example 1, below.

FIG. 3 graphically illustrates data from a cell viability assay comparing an exemplary integrin αvβ3 targeted nanogel loaded with various small molecule kinase inhibitors, as discussed in detail in Example 1, below.

FIG. 4 graphically illustrates data from a cell viability assay comparing the effect of targeting integrin αvβ3 with exemplary nanogels of the invention loaded with docetaxel, as discussed in detail in Example 1, below.

FIG. 5 graphically illustrates data from a cell viability assay comparing exemplary integrin αvβ3 targeted nanogel loaded with taxanes to ABRAXANE™ (paclitaxel), as discussed in detail in Example 1, below.

FIGS. 6A and 6B graphically illustrate data demonstrating that exemplary targeted nanogels of the invention are effective drug delivery vehicles for suppressing breast cancer, as discussed in detail in Example 1, below.

FIGS. 7A and 7B graphically illustrate data demonstrating that exemplary targeted nanogels of the invention as drug delivery vehicles require 15-fold less drug compared to ABRAXANE™, as discussed in detail in Example 1, below.

FIGS. 8A and 8B graphically illustrate data demonstrating that exemplary integrin αvβ3 targeted nanogels of the invention can effectively deliver docetaxel and suppress metastasis, as discussed in detail in Example 1, below.

FIGS. 9A and 9B graphically illustrate data demonstrating that exemplary integrin αvβ3 targeted nanogels of the invention, RGD-Paclitaxel or RGD-Docetaxel, inhibit pancreatic primary tumor growth and metastasis at 15-fold lower dose compared to ABRAXANE™, as discussed in detail in Example 1, below.

It is to be understood that the drawings are for purposes of illustrating the concepts of the invention and are not to scale. Like reference symbols in the various drawings indicate like elements.

### DETAILED DESCRIPTION

In alternative embodiments, the invention provides nanostructures and/or products of manufacture for use as compound delivery vehicles, e.g., as therapeutic (e.g., drug) or diagnostic compound delivery vehicles for e.g., diagnostic, prophylactic and/or therapeutic applications. In one aspect, the invention provides nanostructures or products of manufacture (also called nanoparticles, nanocarriers or nanodevices) having ex vivo or in vivo diagnostic, prophylactic or therapeutic applications. In alternative embodiments, the nanostructures and/or products of manufacture are used in vivo, ex vivo or in vitro (in culture) to deliver a composition, e.g., for targeted delivery of a composition.

In alternative embodiments, the invention provides composite nanoparticles comprising two or more (e.g., several, a plurality of) compartments which in unison function as the composite nanostructure. In one embodiment, the nanoparticles of the invention comprise a combination of a polymer core and a lipid bilayer that interface and incorporate covalently attached lipid-vascular targeting ligands. These composite nanoparticles of the invention can deliver highly effective and selective payloads for therapeutic and/or diagnostic applications.

In alternative embodiments, nanoparticles of the invention act/function as targeted nanoparticle drug (e.g. small molecule or protein), nucleic acid (e.g., siRNA, miRNA) or gene and radionuclide delivery systems. Thus, nanoparticles of the invention can prevent toxic side effects from many drugs, e.g., most anti-cancer or anti-inflammatory drugs, by limiting the need to deliver dosages that cause side effects; e.g., by using nanoparticles of the invention compositions can be administered at low, non-side effect dosages that otherwise would be toxic if administered in pharmaceutically effective dosages by conventional means (e.g., by inhalation, topically, orally or parenterally). In other words, what would be considered a “suboptimal” dosage if delivered by a conventional means can be a pharmaceutically effect amount if delivered in a targeted fashion by using a nanoparticle of the invention.

In another embodiment, a composition (e.g., therapeutic (e.g., drug) or diagnostic) that can be extremely difficult to deliver ex vivo and/or to an individual in vivo, e.g., because of solubility problems (e.g., extreme insolubility in aqueous environments), very limited bioavailability, and the like, can be effectively delivered in a pharmaceutically effect amount if delivered in a targeted fashion by using a nanoparticle of the invention.

In alternative embodiments, nanoparticles of the invention are used to deliver compositions in a targeted manner, e.g., ex vivo and or to an individual in vivo for e.g., the diagnosis, prevention and/or amelioration (e.g., a treatment) of a disease or a condition, e.g., a cancer and/or an inflammatory disease, a toxic exposure (e.g., to a poison or toxin), and the like. In alternative embodiments, nanoparticles of the invention facilitate the specific delivery of agents to any cell or tissue, e.g., a diseased tissues thereby sparing non-diseased tissues.

In alternative embodiments, nanoparticles of the invention are used to deliver nutrients and/or natural products such as ions, metals, chelating agents, lipids, vitamins, minerals, amino acids, carbohydrates, nucleic acids (e.g., DNA or RNA, single or double stranded), polypeptides, peptides and the like.

In alternative embodiments, nanoparticles of the invention are used to deliver diagnostic, prophylactic or therapeutic agents by packaging the active ingredient into a multi-compartmented composite nanostructure. In alternative embodiments, the nanoparticles of the invention comprise a combination of polymer core/lipid bilayer interfaces which incorporate covalently attached lipid-vascular targeting ligands. These can produce highly effective and selective payloads for prophylactic, therapeutic and/or diagnostic strategies.

Biological agents that can be stored in the nanostructures and products of manufacture of this invention include growth factors, collagens, various proteins/biomolecules, genes, enzymes, hormones, nucleic acids (e.g., siRNA, miRNA, and/or single or double-stranded DNA or RNA), antibiotics, drugs, and functional nanoparticles. The nanostructures or products of manufacture of the invention can comprise any desired material, composition or agent, e.g., buffers, pharmaceutically acceptable excipients, drugs, growth factors, hormones, proteins, peptides, enzymes, small molecules, lipids, carbohydrates, sugars, nucleic acids, antibiotics, antibodies, metals, ions, other nanoparticles, nutrients, vitamins and/or minerals.

A nanostructure or product of manufacture of the invention can be delivered in vivo or ex vivo by any means, e.g. through oral doses, inhalation sprays, intraocularly, intravascularly (e.g., intravenously, as by injection), intramuscular injection, topically (as on the mucosa or skin), intradermal, intrathecal or from implanted devices.

For efficient use of drugs where a certain desired therapeutic concentration range is desired to be, or must be, maintained (e.g., as they become ineffective at low levels and are often toxic beyond certain concentrations) the nanostructures or products of manufacture can be used. In one aspect, not all of a dosed drug or other compound (e.g., in nanostructures or products of manufacture of the invention) is delivered to a target, e.g., a targeted organ. Thus, in one aspect, the practicing the invention avoids administrations of a large excess of drug to, make a small amount of drug actually available for the needed therapeutics, which results in deleterious side effects and can also contribute to drug addiction and abuse.

In alternative embodiments, nanoparticles of the invention comprise a polymer central core capable of high loading densities of any desired material, composition or agent with highly selective and/or high affinity ligands which are covalently attached to a lipid monolayer on the surface of the nanoparticle. In alternative embodiments, nanoparticles of the invention comprise ligands such as peptides, organic molecules or antibodies which target angiogenic vasculature, tumor tissue, circulating tumor cells and/or inflammatory cells, or bone marrow derived cells. The target cell or tissue would express the receptor for the ligand present on the nanoparticle; for example, an integrins such as avb3, a4B1 or a5b1, or a non-integrin receptors or cell surface molecules could be targeted.

In one aspect, to Optimize operation of a nanostructure of this invention (e.g., for the delivery of a desired material, composition or agent into a living cell), various materials parameters can are optimized, for example, the sphere size, shell thickness, nanoporosity of the shell, the size of the trapped particles and the like.

Exemplary drug systems that can be incorporated into nanostructures of this invention include a diabetes drug, e.g., an insulin, a hormonal protein, a steroid (e.g., a dexamethasone), an anti-inflammatory or immunosuppressant peptide, polypeptide or steroid hormone, a cancer drug (e.g., paclitaxel, a mitotic inhibitor drug used in cancer chemotherapy), a radio-label or radiotherapeutic agent, and the like. To determine in vivo drug release efficacy, the standard procedure of taking blood (or urine) samples and measure drug metabolites, for example, using the HPLC, can be employed.

In alternative embodiments, a nanostructure of this invention is manufactured (processed) into a nanoparticle in the size range from between about 20 to 1000 nm or from between about 40 to 500 nm.

In one embodiment, a nanostructure of this invention comprises a polymer capable of absorbing hydrophobic and/or hydrophilic molecules. Exemplary polymers that can be used to manufacture (process) a nanostructure of this invention include any polymer capable of forming a hydrogel particle, including various random and block copolymers. Exemplary polymers that can be used to manufacture (process) a nanostructure of this invention also can comprise a crosslinking component, such as an unsaturated moiety capable of ultraviolet (uv) or thermal crosslinking to stabilize the core material. This particle can serve as a core for further elaboration into a targeted delivery system by coating it with any one or several of a variety of lipids.

In alternative embodiments, nanostructures of the invention comprise lipids or phospholipid covalently modified with one or a variety of ligands, e.g., a ligand or ligands capable of binding to an integrin such as avb3, a4b1 a5b1 and/or plaktin, and the like. The ligand can be either a linear peptide, cyclic peptide or synthetic organic molecule mimicking these peptides; e.g., cilengitide (based on the cyclic peptide cyclo(-RGDfV-), which is selective for αᵥ integrin. In alternative embodiments, these ligands also can also be antibodies, e.g., monoclonal antibodies, e.g., VITAXIN™ (MedImmune, Gaithersburg, Md.) (a humanized monoclonal antibody against the vascular integrin αᵥβ₃) or M-200.

In alternative embodiments, nanostructures of the invention comprise a second lipid or phospholipid covalently attached to a variety of water soluble oligomers of polymers, e.g., of the following generic class of compounds: ethyleneglycol, vinyllpyrollidone, vinyl alcohol or polysaccharides and the like. In one embodiment, this second lipid acts as a stealth component on the monolayer to improve biological compatibility and increase in vivo circulation rates.

In alternative embodiments, nanostructures of the invention comprise a third component comprising a stabilizing molecule, such as cholesterol or cholesteric analog, or a crosslinkable lipid containing UV sensitive. This component can be sandwiched between the lipid components to increase the melting range of the monolayer component or a mildly crosslinked lipid to stabilize the liposome layer.

In alternative embodiments, nanostructures of the invention comprise a fourth component comprising a bioactive material such as an anticancer cytostatic or cytotoxic agent, as for example gemcitabine or a small molecule inhibitor of oncogenic or inflammatory proteins, such as a kinase inhibitor. Alternatively the bioactive component can be a cytotoxic lipopeptide such as somocystinamide, or curacin A.

In alternative embodiments, nanostructures of the invention comprise therapeutic agents for the treatment of cancer, e.g., a monoclonal antibody, a peptide, a synthetic polypeptide or peptidomimetic, a nucleic acid, a synthetic nucleic acid, a lipid, a carbohydrate and/or a small molecule. The therapeutic agent for the treatment of cancer can comprise or consist of sorafenib (NEXAVAR™), sunitinib (e.g., SUTENT)™, erlotinib (e.g., TARCEVA™), imatinib (e.g., GLEEVEC™), lapatinib (e.g., TYKERB™), bevacizumab (e.g., AVASTIN™), trastuzumab (e.g., HERCEPTIN™), cetuximab (e.g., ERBITUX™), bevacizumab (e.g., AVASTIN™), BIBW 2992, gefitinib (e.g., IRESSA™), ranibizumab (e.g., LUCENTIS™), pegaptanib (e.g., MACUGEN™), dasatinib (e.g., BMS-354825™), sunitinib (e.g., SUTENT)™, pazopanib, nilotinib (e.g., TASIGNA™), panitumumab (e.g., VECTIBIX™), bandetinib, brivanib, E7080™ or a combination thereof.

In alternative embodiments, nanostructures of the invention comprise protein kinase inhibitors, e.g., a tyrosine kinase inhibitor or a serine/threonine kinase inhibitor.

In alternative embodiments, nanostructures of the invention comprise angiogenesis inhibitors, e.g., a vascular endothelial growth factor (VEGF)-mediated angiogenesis inhibitor.

In alternative embodiments, nanostructures of the invention comprise inducers of apoptosis or a mitotic and anti-microtubule inhibitor (inhibition of microtubule function), e.g., a raltitrexed (e.g., TOMUDEX™), doxorubicin (e.g., ADRIAMYCIN™), fluorouracil (e.g., 5-fluorouracil), paclitaxel (e.g., TAXOL™ or ABRAXANE™) docetaxel (e.g., TAXOTERE™), vinblastin, vindesine, vinorelbine (NAVELBINE™); an epothilone (e.g., epothilone A, B, C, D, E or F), ixabepilone (also known as azaepothilone B, e.g., BMS-247550™) or a combination thereof.

In alternative embodiments, nanostructures of the invention comprise an alkylating agent, e.g., a cisplatin, cisplatinum or cis-diamminedichloridoplatinum(II) (CDDP), carboplatin, oxaloplatin, cyclophosphamide (cytophosphane) (e.g., ENDOXAN™, CYTOXAN™, NEOSAR™, REVIMMUNE™), mechlorethamine (chlormethine, mustine, nitrogen mustard), chlorambucil (e.g., LEUKERAN™) or a combination thereof.

In alternative embodiments, nanostructures of the invention comprise a topoisomerase inhibitor. e.g., an etoposide (e.g. EPOSIN™. ETOPOPHOS™, VEPESID™, VP-16™), amsacrine, topotecan HYCAMTIN™), teniposide (e.g., VUMON™. VM-26™), an epipodophyllotoxin, a camptothecin, irinotecan (e.g., CAMPTOSAR™), or a combination thereof.

In alternative embodiments, nanostructures of the invention comprise a glycopeptide antibiotic, e.g., a bleomycin (e.g., bleomycin A₂ or B₂), mitomycin (e.g., mitomycin C), plicamycin (also known as mithramycin; e.g. MITHRACIN™), or a combination thereof.

In alternative embodiments, nanostructures of the invention comprise a steroid receptor inhibitor or steroid inhibitor (an anti-steroid), e.g., an estrogen receptor modulator (a SERM), such as a tamoxifen (e.g., NOLVADEX™, ISTUBAL™ VALODEX™); or, the steroid inhibitor or an anti-steroid can comprise or consist of a finasteride (e.g., PROSCAR™, PROPECIA™, FINCAR™, FINPECIA™, FINAX™, FINAST™, FINARA™, FINALO™, PROSTERIDE™, GEFINA™, APPECIA™, FINASTERID IVAX™, FINASTERID ALTERNOVA™).

In alternative embodiments, nanostructures of the invention comprise a matrix metalloproteinase (MMP) inhibitor.

Exemplary covalent bonds by which the targeting moieties are associated with the nanocarriers of the invention include, for example, amide (—CONH—); thioamide (—CSNH—); ether (ROR′), where R and R′ may be the same or different and are other than hydrogen; ester (—COO—); thioester (—COS—); —O—; —S—; —Sₙ, where n is greater than 1, preferably approximately 2 to approximately 8, and more preferably approximately 2; carbamates; —NH—; —NR—, where R is alkyl, for example, alkyl of from 1 carbon to approximately 4 carbons; urethane; and substituted imidate; and combinations of two or more of these. Covalent bonds between targeting ligands and polymers may be achieved through the use of molecules that may act as spacers to increase the conformational and topographical flexibility of the ligand. Examples of such spacers include, for example, succinic acid, 1,6-hexanedioic acid, 1,8-octanedioic acid, and the like, as well as modified amino acids, such as, for example, 6-aminohexanoic acid, 4-aminobutanoic acid, and the like. In addition, in the case of targeting ligands which comprise peptide moieties, side-chain-to-side-chain cross-linking may be complemented with side chain-to-end cross-linking and/or end-to-end cross-linking. Also, small spacer molecules, such as dimethylsuberimidate, may be used to accomplish similar objectives. The use of agents, including those used in Schiff's base-type reactions, such as gluteraldehyde, may also be employed.

The covalent linking of targeting moieties to a composite structure of the invention also can be accomplished using synthetic organic techniques. For example, the targeting moieties may be linked to the materials, including the polymers, via the use of well-known coupling or activation agents, e.g., activating agents, e.g., electrophilic activating agents can be employed to elicit the formation of a covalent bond. Exemplary activating agents which may be used include, for example, carbonyldiimidazole (CDI), dicyclohexylcarbodiimide (DCC), diisopropyl-carbodiimide (DIC), methyl sulfonyl chloride, Castro's Reagent, and diphenyl phosphoryl chloride. The covalent bonds may involve cross-linking and/or polymerization. In alternative embodiments, cross-linking includes attachment of two chains of polymer molecules by bridges comprising e.g., an element, a group, or a compound, which join certain carbon atoms of the chains by covalent chemical bonds. For example, cross-linking may occur by photopolymerization and for polypeptides which are joined by the disulfide bonds of the cysteine residue. Cross-linking may be achieved, for example, by (1) adding a chemical substance (e.g., cross-linking agent) and exposing the mixture to heat, or (2) subjecting a polymer to high-energy radiation. A variety of cross-linking agents, or “tethers,” of different lengths and/or functionalities can be used.

In alternative embodiments, composite nanostructures of the invention comprise phosphatidylcholines such as dioleoylphosphatidylcholine, dimyristoylphosphatidylcholine, dipalmitoylphosphatidylcholine (DPPC), and distearoylphosphatidylcholine; phosphatidylethanolamines such as dipatmitoylphosphatidylethanolamine (DPPE), dioleoylphosphatidylethanolamine, and N-succinyl-dioleoylphosphatidylethanolamine; phosphatidylserines, phosphatidylglycerols, and sphingolipids; glycolipids such as ganglioside GM1; glucolipids, sulfatides, and glycosphingolipids; phosphatidic acids such as dipalmitoylphosphatidic acid (DPPA); palmitic fatty acids, stearic fatty acids, arachidonic fatty acids, lauric fatty acids, myristic fatty acids, lauroleic fatty acids, physeteric fatty acids, myristoleic fatty acids, palmitoleic fatty acids, petroselinic fatty acids, oleic fatty acids, isolauric fatty acids, isomyristic fatty acids, isopatmitic fatty acids, and isostearic fatty acids; cholesterol and cholesterol derivatives such as cholesterol hemisuccinate, cholesterol sulfate, and cholesteryl-(4′-trimethylammonio)-butanoate; polyoxyethylene fatty acid esters, polyoxyethylene fatty acid alcohols, polyoxyethylene fatty acid alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene fatty acid polymers, polyoxyethylene fatty acid stearates, 12-(((7′-diethylaminocoumarin-3-yl)-carbonyl)-methylamino)-octadecanoic acid, N-(12-(((7′-diethylamino-coumarin-3-yl)-carbonyl)-methyl-amino)octa-decanoyl)-2-amino-palmitic acid, 1,2-dioleoyl-sn-glycerol, 1,2-dipalmitoyl-sn-3-succinylglycerol, 1,3-dipalmitoyl-2-succinyl-glycerol, 1-hexadecyl-2-palmitoyl-glycerophosphoethanolamine, and palmitoylhomocysteine, lauryltrimethylammonium bromide (lauryl-=dodecyl-); cetyltrimethylammonium bromide (cetryl-=hexadecyl-), myristyltrimethylammonium bromide (myristyl-=tetradecyl-); alkylmethylbenzylammonium chlorides such as wherein alkyl is a C.sub.12, C.sub.14, or C.sub.1-6 alkyl; and benzyldimethyldodecylammonium bromide, benzyldimethyldodecylammonium chloride, benzyldimethylhexadecylammonium bromide, benzyldimethylhexadecylammonium chloride, benzyldimethyltetradecylammonium bromide, benzyldimethyltetradecylammonium chloride, cetyldimethylethylammonium bromide, cetyldimethylethylammonium chloride, cetylpyridinium bromide, cetylpyridinium chloride, N-(1-2,3-dioleoyloxy)-propyl)-N,N,N-trimethylammonium chloride (DOTMA), 1,2-dioleoyloxy-3-(trimethylammonio)propane (DOTAP), and 1,2-dioleoyl-c-(4′-trimethylammonio)-butanoyl-sn-glycerol (DOTB). Any one or combination of these compositions (e.g., lipids) can be used in the first lipid or phospholipid layer or coating over (substantially covering) the polymer core of a composition of this invention.

In alternative embodiments, composite nanostructures of the invention comprise compositions to target a specific cell, cell type, antigen, cellular membrane protein, organ, tissue markers, tumor marker, angiogenesis marker, blood vessel, thrombus, fibrin and/or infective agent. In alternative embodiments, composite nanostructures of the invention comprise as targeting moieties ligands for adhesion molecules (e.g., integrin as αv/β3), intercellular adhesion molecule-1 (1-CAM-1), fibrinogen receptor GPIIb/IIIa, VEGF receptors and/or any receptor expressed in regions of angiogenesis, inflammation, or thrombus. In alternative embodiments, composite nanostructures of the invention comprise as targeting moieties complementary receptor ligands, targeting ligands, proteins, and fragments thereof. In alternative embodiments, composite nanostructures of the invention comprise compositions, e.g., ligands, to target a cell, e.g., endothelial cells, neoplastic cells, and blood cells, including targeting VEGFR2, I-CAM-1, alpha-v/beta3 integrin, alpha-v/integrin, fibrinogen receptor GPIIb/IIIa, P-selectin and/or mucosal vascular addressin cell adhesion molecule-1.

In alternative embodiments, composite nanostructures of the invention comprise antibodies, e.g., monoclonal antibodies to target a specific cell, cell type, antigen, cellular membrane protein, organ, tissue markers, tumor marker, angiogenesis marker, blood vessel, thrombus, fibrin and/or infective agent. For example, exemplary antibodies can bind to tumor marker proteins, organ or cell type specific markers, or infective agent markers. In alternative embodiments, composite nanostructures of the invention are targeted using antibodies, proteins, fragments thereof, aptamers, or other ligands, e.g., to sites of neoplasia, angiogenesis, thrombus, inflammation, infection, to diseased or normal organs or tissues, e.g., targeted to blood, heart, brain, blood vessel, kidney, muscle, lung, and liver, and/or to extracellular or transmembrane proteins. The targeted markers, including tumor markers, can be the extracellular domain of a protein. The antibodies or fragments thereof can be designed to target these marker proteins can bind to any portion of the protein. The antibodies can bind to the extracellular portion of a protein, for example, a cellular transmembrane protein. Antibodies, proteins, and fragments thereof can be used to specifically or selectively target a desired target molecule.

In alternative embodiments, targeting moieties are bound or “complexed” to a composite nanostructures of the invention by covalent or noncovalent associations, for example, using diethylenetriaminepentaacetic acid (DTPA); ethylenediaminetetraacetic acid (EDTA); 1,4,7,10-tetraazacyclododecane-N,N′N″,N′″-tetraacetic acid (DOTA); 1,4,7,10-tetraazacyclododecane-N,N′,N″-triacetic acid (DOTA); 3,6,9-triaza-12-oxa-3,6,9-tricarboxymethylene-10-carboxy-13-phenyltrideca-noic acid (B-19036); hydroxybenzylethylenediamine diacetic acid (HBED); N,N′-bis(pyridoxy)-5-phosphate)ethylene diamine; N,N′-diacetate (DPDP); 1,4,7-triazacyclononane-N,N′,N″-triacetic acid (NOTA); 1,4,8,11-tetraazacyclotetradecane-N,N′,N″,N′″-tetraacetic acid (TETA); kryptands (macrocyclic complexes); or desferrioxamine. Other exemplary complexing agents comprise EDTA, DTPA, DOTA, DO3A, a kryptand or a DTPA. Other exemplary complexing agents comprise N,N′-bis-(carboxydecylamidomethyl-N-2,3-dihydroxypropyl)ethylenediamine-N—,N′-diacetate (EDTA-DDP); N,N′-bis-(carboxyoctadecylamidomethyl-N-2,3-dihydroxypropyl)ethylenediami-ne-N,N′-diacetate (EDTA-ODP); and N,N-Bis(carboxylaurylamidomethyl-N-2,3-dihydroxypropyl)ethylenediamine-N—,N′-diacetate (EDTA-LDP). Other exemplary binding/complexing agents comprise albumin, collagen, polyarginine, polylysine, polyhistidine, gamma-globulin or beta-globulin with albumin, polyarginine, polylysine or polyhistidine. Other exemplary binding/complexing agents comprise Mn(II)-DTPA, Mn(II)-EDTA, Mn(II)-DOTA, Mn(II)-DO3A, Mn(II)-kryptands, Gd(III)-DTPA, Gd(III)-DOTA, Gd(III)-DO3A, Gd(III)-kryptands, Cr(III)-EDTA, Cu(II)-EDTA, or iron-desferrioxamine.

In alternative embodiments, composite nanostructures of the invention comprise for targeted delivery nucleic acids such as RNA and DNA of either natural or synthetic origin, recombinant RNA and DNA, antisense RNA, microRNAs (miRNAs), short hairpin RNAs (shRNAs), RNA interference (RNAi), and small interfering RNA (siRNA), including other small RNA-based therapeutics); expression vectors such as plasmids, phagemids, cosmids, yeast artificial chromosomes (YACs), defective or “helper” viruses, viral subcomponents, viral proteins or peptides, either alone or in combination with other agents; antisense nucleic acids, including single- and/or double-stranded RNA and DNA, and analogs thereof such as phosphorothioate and/or phosphorodithioate oligo-deoxynucleotides.

In alternative embodiments, composite nanostructures of the invention comprise for targeted delivery peptides, polypeptides, and proteins such as adrenocorticotropic hormone, angiostatin, Angiotensin Converting Enzyme (AGE) inhibitors (e.g., captopril, enalapril, and lisinopril), bradykinins; calcitonins, cholecystokinins, and collagenases; enzymes such as alkaline phosphatase and cyclooxygenases colony stimulating factors, corticotropin release factor, dopamine, elastins, epidermal growth factors, erythropoietin, transforming growth factors, fibroblast growth factors, glucagon, glutathione, granulocyte colony stimulating factors, granulocyte-macrophage colony stimulating factors, human chorionic gonadotropin, IgA, IgG, IgM, inhibitors of bradykinins, insulin, integrins, interferons (e.g., interferon alpha, interferon beta, interferon gamma); ligands for Effector Cell Protease Receptors, thrombin, manganese super oxide dismutase, metalloprotein kinase ligands, oncostatin M, interleukins (e.g. interleukin 1, interleukin 2, interleukin 3, interleukin 4, interleukin 5, interleukin 6, interleukin 7, interleukin 8, interleukin 9, interleukin 10, interleukin 11, and interleukin 12), opiate peptides (e.g., enkephalines and endorphins); and oxytocin, pepsins, platelet-derived growth factors, lymphotoxin, promoters of bradykinins, Protein Kinase C, streptokinase, substance P (i.e., a pain moderation peptide), tissue plasminogen activator, tumor necrosis factors, nerve growth factors, urokinase, vascular endothelial cell growth factors and/or vasopressin.

In alternative embodiments, composite nanostructures of the invention comprise for targeted delivery: 15-deoxy spergualin, 17-alpha-acyl steroids, 3-(Bicyclyl methylene) oxindole, 3 alpha-, 5 alpha-tetrahydrocortisol, 5 alpha-reductase inhibitor, adaprolol enantiomers, aldose reductase inhibitors (e.g., sorbinil and tolrestat), aminoguanidine, antiestrogenics (e.g., 24-(1,2-diphenyl-1-butenyl)phenoxy)-N,N-dimethylethanamine) apraclonidine hydrochloride, aurintricarboxylic acid, azaandrosterone, bendazac, benzoylcarbinol salts, betaxolol, bifemelane hydrochloride, bioerodible poly(ortho ester), cetrorelix acetate, cidofovir, vitamin E, dipifevrin, dipyridamole+aspirin, dorzolamide, epalrestat, etofibrate, etoposide, filgastrim, foscarnet, fumagillin, ganciclovir, granulocyte macrophage colony stimulating factor (GM-CSF), haloperidol, imidazo pyridine, latanoprost, lecosim, levobunolol, N-4 sulphanol benzyl-imidazole, N-acyl-5-hydroxytryptamine, nipradilol, nitric oxide synthase inhibitors, pilocarpine, ponalrestat, prostanoic acid, S-(1,3 hydroxyl-2-phosphonylmethoxypropyl) cytosine, somatuline, sorvudine, ticlopidine, timolol, vaminolol, vascular endothelial growth factor, and/or alpha-interferon.

In alternative embodiments, composite nanostructures of the invention comprise for targeted delivery anti-inflammation agents; amelexanox; anti-anginals such as diltiazem, erythrityl tetranitrate, isosorbide dinitrate, nifedipine, nitroglycerin (glyceryl trinitrate), pentaerythritol tetranitrate, and verapamil; antibiotics such as amoxicillin, ampicillin, bacampicillin, carbenicillin, cefaclor, cefadroxil, cephalexin, cephradine, chloramphenicol, clindamycin, cyclacillin, dapsone, dicloxacillin, erythromycin, hetacillin, lincomycin, methicillin, nafcillin, neomycin, oxacillin, penicillin G, penicillin V, picloxacillin, rifampin, tetracycline, ticarcillin, and vancomycin hydrochloride; anti-coagulants such as phenprocoumon, and heparin; anti-fungal agents such as polyene antibiotics like flipin, natamycine, and rimocidin; imidazoles such as clotrimazole, ketoconazole, and micronazole; triazoles such as fluconazole, itraconazole, and ravuconazole; allylamines such as amorolfin, butenafine, naftifine, and terbinafine; echinocandins such as caspofungin, micafungin, and ulafungin, and others such as amphotericin B, flucytosine, griseofulvin, miconazole, nystatin, and ricin; anti-inflammatories such as aspirin difimisal, ibuprofen, indomethacin, meclofenamate, mefenamic acid, naproxen, oxyphenbutazone, phenylbutazone, piroxicam, salicylates, sulindac, and tolmetin; anti-neoplastic agents such as adriamycin, aminoglutethimide, amsacrine (m-AMSA), ansamitocin, arabinosyl adenine, arabinosyl, asparaginase (L-asparaginase), Erwina asparaginase, bisitnidazoacridones, bleomycin sulfate, bleomycin, bleomycin, busulfan, carzelesin, chlorambucil, cytosine arabinoside, dactinomycin (actinomycin D), daunorubicin hydrochloride, doxorubicin hydrochloride, estramustine phosphate sodium, etoposide (VP-16), flutamide, interferon alpha-2a, interferon alpha-2b, leuprolide acetate mercaptopolylysine, leuprolide acetate, megestrol acetate, melphalan (e.g., L-sarolysin or phenylalanine mustard), mercaptopurine, methotrexate, methotrexate, mitomycin, mitomycin, mitotane, platinum compounds (e.g., spiroplatin, cisplatin, and carboplatin), plicamycin (mithramycin), procarbazine hydrochloride, tamoxifen citrate, taxol, teniposide (VM-26), testolactone, trilostane, vinblastine sulfate (VLB), vincristine sulfate, and vincristine; anti-protozoans such as chloroquine, hydroxychloroquine, metronidazole, quinine, and meglumine antimonate; anti-rheumatics such as penicillamine; and anti-virals such as abacavir, acyclovir, amantadine, didanosine, emtricitabine, enfuvirtide, entecavir, ganciclovir, gardasil, lamivudine, nevirapine, nelfinavir, oseltamivir, ribavirin, rimantadine, ritonavir, stavudine, valaciclovir, vidarabine, zalcitabine, and zidovudine.

In alternative embodiments, composite nanostructures of the invention comprise for targeted delivery a biological response modifiers such as muramyldipeptide, muramyltripeptide, prostaglandins, microbial cell wall components, lymphokines (e.g., bacterial endotoxin such as lipopoly saccharide, macrophage activation factor, etc.), and bacterial polypeptides such as bacitracin, colistin, and polymixin B; blood products such as parenteral iron, hemin, hematoporphyrins, and their derivatives; cardiac glycosides such as deslanoside, digitoxin, digoxin, digitalin, and digitalis; and circulatory drugs such as propranolol.

In alternative embodiments, composite nanostructures of the invention comprise for targeted delivery visualizing agents, e.g., dyes such as fluorescent dyes and colorimetric dyes, e.g., 3HCl, 5-carboxyfluorescein diacetate, 4-chloro-1-naphthol, 7-amino-actinomycin D, 9-azidoacridine, acridine orange, allophycocyanin, amino methylcoumarin, benzoxanthene-yellow, bisbenzidide H 33258 fluorochrome, BODIPY FL, BODIPY TMR, BODIPY-TR, bromocresol blue, bromophenol blue, carbosy-SNARF, Cascade blue, chromomycin-A3, dansyl+R—NH.sub.2, DAPI, DTAF, DTNB, ethidium bromide, fluorescein, fluorescein-5-maleimide diacetate, FM143, fura-2, Indo-1, lucifer yellow, methylene blue, mithramycin A, NBD, oregon green, propidium, iodide, rhodamine 123, rhodamine red-X, R-Phycoerythrin, SBF1, SIST, sudan black, tetramethyl purpurate, tetramethylbenzidine, tetramethylrhodamine, texas red, thiazolyl blue, TRITC, YOYO-1, and the like; fluorescein isothiocyanate, fluorescein isothiocyanate albumin, fluorescein isothiocyanate antibody conjugates, fluorescein isothiocyanate alpha-bungarotoxin, fluorescein isothiocyanate-casein, fluorescein isothiocyanate-dextrans, fluorescein isothiocyanate-insulin, fluorescein isothiocyanate-lectins, fluorescein isothiocyanate-peroxidase and/or fluorescein isothiocyanate-protein A.

In alternative embodiments, composite nanostructures of the invention comprise for targeted delivery anesthetics such as droperidol, etomidate, fentanyl citrate with droperidol, ketamine hydrochloride, methohexital sodium, and thiopental sodium, and/or radioactive particles or ions such as strontium, iodide rhenium, technetium, cobalt and/or yttrium.

In alternative embodiments, composite nanostructures of the invention comprise for targeted delivery bioactive agents such as monoclonal antibodies or monoclonal antibody fragments.

In alternative embodiments, composite nanostructures of the invention comprise for targeted delivery growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunsolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide, fludrocortisone acetate, progesterone, testosterone, and adrenocorticotropic hormone; local anesthetics such as bupivacaine hydrochloride, chloroprocaine hydrochloride, etidocaine hydrochloride, lidocaine hydrochloride, mepivacaine hydrochloride, procaine hydrochloride, and tetracaine hydrochloride; metabolic potentiators such as glutathione; antituberculars such as para-aminosalicylic acid, isoniazid, capreomycin sulfate cycloserine, ethambutol hydrochloride ethionanide, pyrazinamide, rifampin, and streptomycin sulfate; narcotics such as paregoric, and opiates such as codeine, heroin, methadone, morphine, and opium; neuromuscular blockers such as atracurium besylate, gallamine triethiodide, hexafluorenium bromide, metocurine iodide, pancuronium bromide, succinylcholine chloride (suxamethonium chloride), tubocurarine chloride, and vecuronium bromide; sedatives (i.e., hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam, and triazolam; subunits of bacteria (e.g., Mycobacteria, Corynebacteria), the synthetic dipeptide N-acetyl-muramyl-L-alanyl-D-isoglutamine, and the like; and/or vitamins such as cyanocobalamin neinoic acid, retinoids and derivatives such as retinol palmitate, alpha-tocopherol, naphthoquinone, cholecalciferol, folic acid, and tetrahydrofolate.

The invention will be further described with reference to the following examples; however, it is to be understood that the invention is not limited to such examples.

### EXAMPLES

### Example 1

### Formation of Exemplary Compositions of the Invention: Nanogels

The following example describes exemplary nanogels of the invention and exemplary methods for making them.

Formation of Nanogels. Using the liposome as a “template” to encapsulate proteins and polymers for further crosslinking, we encapsulate albumin-bound docetaxel and UV crosslink the core to form lipid coated nanogels. The liposome is an ideal template since it can be extruded to the desired hydrodynamic diameter (100 nm) and it can easily incorporate targeting ligands such as cRGDfK and surface modifications such as polyethylene glycol (PEG) coatings, provided that they are conjugated to lipid.

The nanoparticle formulation of Cholesterol:DOPE:DSPC:DSPE-(PEO)₄-cRGDfK:DSPE-mPEG2000 (6:6:6:1:1 molar ratio) in chloroform were evaporated under argon gas and the dried lipid film was hydrated with a solution containing the desired monomers (HEMA/PEG, albumin, casein) with drug and a photo-initiator such as IRGACURE 2959™ in a total volume of 5 ml at a total lipid concentration of 3.3 mM. Liposomes were vortexed for 2-3 minutes to remove any adhering lipid film and sonicated in a bath sonicator (ULTRASONIK™ (ULTRAsonik) 28×) for 2-3 min at room temperature to produce multilamellar vesicles (MLV). MLVs were then sonicated with a Ti-probe (BRANSON 450™ sonifier) for 1-2 minutes to produce small unilamellar vesicles (SUVs) as indicated by the formation of a translucent solution. To reduce the size of the SUVs, stepwise extrusion was performed with the final step being extrusion through a polycarbonate filter with 100 nm pore size (Whatman). The nanoparticles were purified on SEPHAROSE CL-4B™ columns to remove free monomer and drug. After the column step, the nanoparticles were exposed to UV light at 365 nm for 5 min to crosslink the core, producing the final nanogel. Scanning electron micrographs of these particles demonstrated an average hydrodynamic diameter of 100 nm (also confirmed by dynamic light scattering) and we observed both a bilayer and a dense nanogel core as expected.

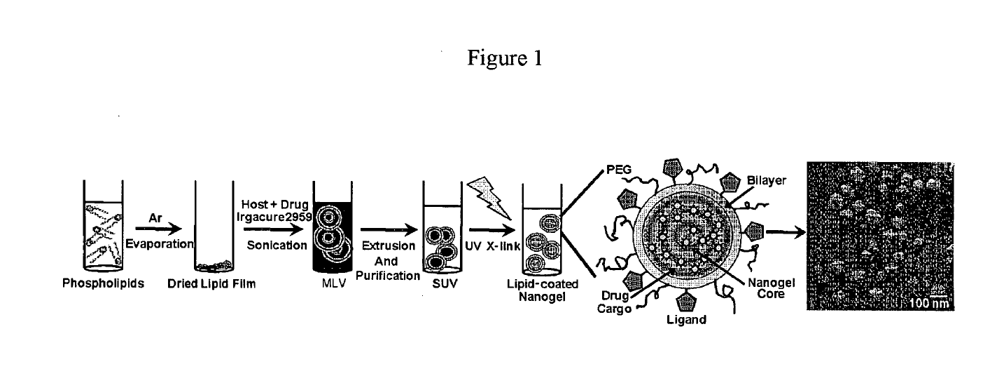

FIG. 1 illustrates a schematic of an exemplary method for preparing exemplary nanogels of the invention using the exemplary method comprising extrusion and UV crosslinking with a photoinitiator (IRGACURE 2959™). This method allows for inclusion of targeting ligands and surface coatings in the lipid bilayer template and enables loading of various cargoes by fine tuning the nanogel core to load the specific cargo. The core can be loaded with a wide variety of cargoes including both hydrophobic and hydrophilic small molecule drugs, nucleic acids, peptides, and other biologicals. The core can be a mixture of monomeric inputs including but not limited to: human serum albumin, casein, hydroxypropyl-β-cyclodextrin, hydroxyethyl methacrylate (HEMA), and polyethylene glycol (PEG). The scanning electron micrograph to the right demonstrates the homogeneity of nanogels made of a lipid bilayer featuring a PEG2000™ coating, cRGDfK targeting peptide, and a core consisting of docetaxel bound to human serum albumin, which has been UV crosslinked. Scale bar is 100 nm.

Drug Loading and Quantitation. The amount of encapsulated drug was quantified by adding 1.5% TRITON X-100™ to disrupt the nanoparticles and comparing the absorbance at various wavelengths on a spectrophotometer to a standard curve of free drug. The wavelengths used for bortezomib, sunitinib, sorafenib, 17-AAG, dasatinib, bosutinib, paclitaxel, and docetaxel were 270, 270, 270, 330, 260, 310, 227, and 227 nm, respectively. All compounds were purchased from Chemietek, Inc. For paclitaxel and docetaxel, HPLC (Gilson) quantification was performed using Trilution software to integrate the area under the curve. The taxanes were extracted from the nanogels and analyzed on a Luna 5 μm PFP column (Phenomenex) using isocratic mode, 60:40 acetonitrile:water at 1 ml/min. Dual detection was used at both 227 and 280 nm. The area under the curve for each sample was compared to a standard curve of free drug.

Cell viability assays. For XTT assays, cells were grown in 96-well plates overnight and all assays were conducted in growth medium will full serum and additives. Nanoparticles were serially diluted into medium to give the appropriate concentration of loaded drug and incubated with the cells at 4° C. for 10 min. The nanoparticles were washed away and replaced with fresh medium and returned to 37° C. for 72 hours (h). At this time point, cell viability was quantified at 450 nm after the addition of 1 mg/ml XTT solution (Sigma-Aldrich) in phenol-red free DMEM medium containing phenoxymethosulfate (Sigma-Aldrich). Dose-response curves were plotted using GRAPHPAD PRISM™ (GraphPad Prism) software and EC₅₀s were calculated using this program.

Orthotopic Pancreatic Cancer Model. The orthotopic pancreatic carcinoma model has been previously described (28, 29). Briefly, 6-10 week old nude mice were injected with 1 million syngeneic murine R40P (isolated from a spontaneous pancreatic tumor from the genetically engineered mouse model: Pdx1-Cre/LSL-Kras^{G12D}/Ink4a/Arf^{lox/lox}) cells in the tail of the pancreas. Nanogels containing either docetaxel or paclitaxel were injected intravenously on day 5, 7, and 9 post-surgical implantation of the cells. On day 11, the primary tumor as well as the hepatic hilar lymph node were resected and weighed.

In a second experiment, 250,000 R40P cells were injected in the pancreas and the mice were dosed on day 7, 9, 11, 13, 15, 17, and 19 with harvesting of the tumors on Day 21. To quantify metastasis, the combined weight of both the hepatic hilar lymph node and metastatic lesions was measured. ABRAXANE™ (Abraxis Bioscience, Los Angeles, Calif.) was resuspended in sterile saline and dosed intravenously based on its paclitaxel content at 1, 5, or 15 mg/kg on an identical qod schedule.

Orthotopic Breast Carcinoma Model. MDA-MB-231/Lm-2 cells were derived from metastatic sites in the lung in an orthotopic breast cancer model as described by Kerbel and colleagues. Briefly, the mammary fat pad (#4) was injected with 2 million cells and tumors were allowed to grow to the size of ˜70-80 mm³. Nanoparticle dosing was initiated via iv (IV) administration on a god schedule. The breast tumors were measured with calipers after drug administration every other day and tumor size was calculated using the formula: Size=(Length*(Width)²)/2. The animals were sacrificed once control tumors reached 1000 mm³. At this time, the primary tumors were resected and weighed for comparison to the size measurements. Drug doses were the same as the orthotopic pancreatic carcinoma.

Statistical analysis. Error bars represent mean values±s.e.m. The statistical significance of the experiments was determined using a two-tailed Student's t-test; p values <0.05 were considered significant.

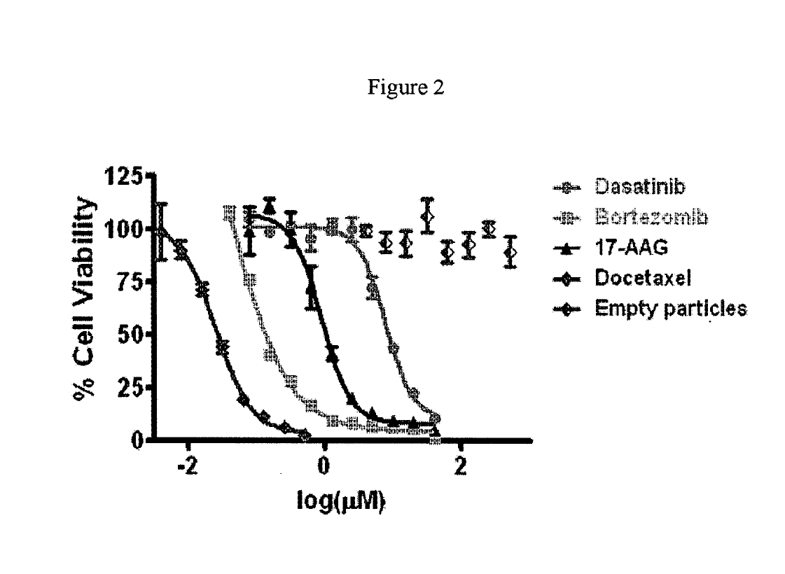

FIG. 2 graphically illustrates data from a cell viability assay comparing an exemplary integrin αvβ3 targeted nanogel loaded with various small molecules. The nanogels were prepared with human serum albumin carrying the various drugs as the core component which is crosslinked. The assays are done with M21 cells which express αvβ3. Nanogels are exposed to the cells at 4° C. for 10 min before being washed away. The cells are then returned to 37° C. for 72 h and cell viability is measured with XTT at 450 nm. EC50s, calculated using GRAPHPAD PRISM™, are listed in Table 1, below. Error bars represent ±s.e.m.

In summary, the data in FIG. 2 demonstrates that these exemplary nanogels of the invention, made with human serum albumin in the core, can carry multiple chemotypes and inhibit cell viability, demonstrating their ability to deliver the drugs in vitro and in vivo.

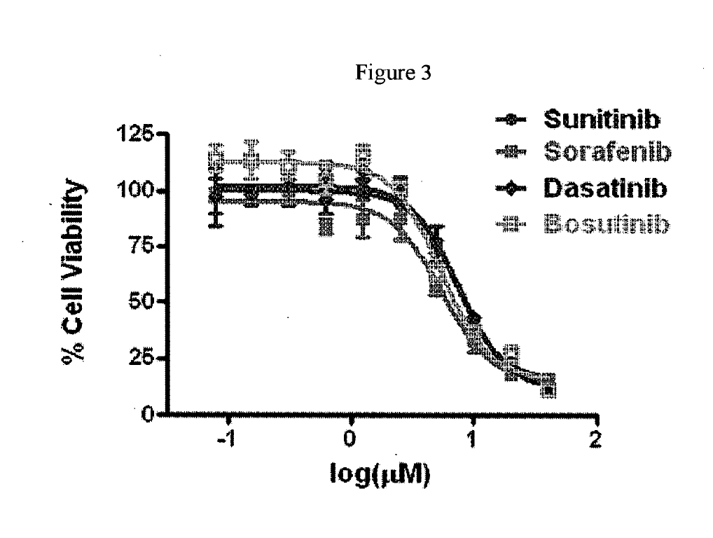

FIG. 3 graphically illustrates data from a cell viability assay comparing an exemplary integrin αvβ3 targeted nanogel loaded with various small molecule kinase inhibitors. The nanogels were prepared with human serum albumin carrying the various drugs as the core component which is crosslinked. The assays are done with M21 cells which express αvβ3. Nanogels are exposed to the cells at 4° C. for 10 min before being washed away. The cells are then returned to 37° C. for 72 h and cell viability is measured with XTT at 450 nm. EC50s, calculated using GRAPHPAD PRISM™, are listed in Table 1, below. Error bars represent ±s.e.m.

In summary, the data in FIG. 3 demonstrates that these exemplary nanogels of the invention, made with human serum albumin in the core, can carry multiple kinase inhibitors that inhibit cell viability. This demonstrates their ability to deliver the drugs in vitro and in vivo and that the drug cargo is efficacious.

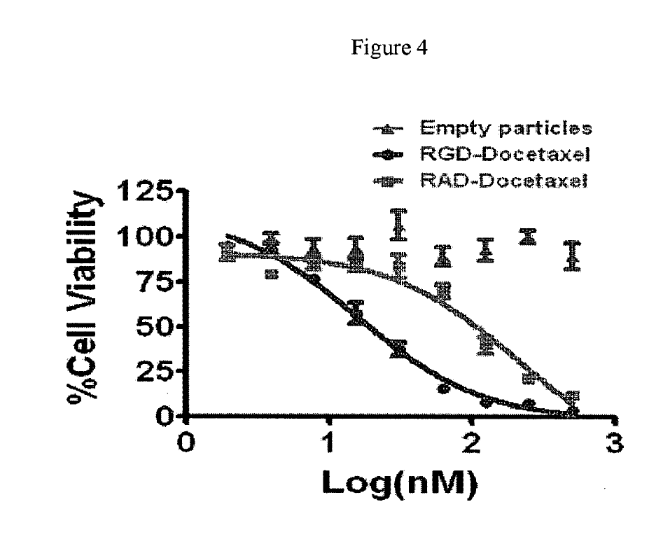

FIG. 4 graphically illustrates data from a cell viability assay comparing the effect of targeting integrin αvβ3 with exemplary nanogels of the invention loaded with docetaxel. The assays are done with M2.1 cells which express αvβ3. Nanogels are exposed to the cells at 4° C. for 10 min before being washed away. The cells are then returned to 37° C. for 72 h and cell viability is measured with XTT at 450 nm. RAD is the control untargeted nanogel whereas RGD is the nanogel targeted to integrin αvβ3. The empty targeted nanogels show no effect on cell viability. EC50s are 16 and 204 nM for RGD-docetaxel and RAD-docetaxel, respectively. Error bars represent ±s.e.m.

In summary, the data in FIG. 4 demonstrates that these exemplary nanogels of the invention targeting integrin αvβ3 and carrying albumin bound docetaxel increase their efficacy over untargeted nanogels (RAD). This supports the targeting mechanism for enhancing efficacy. Secondly, nanogels without docetaxel, but with albumin do not inhibit cell viability.

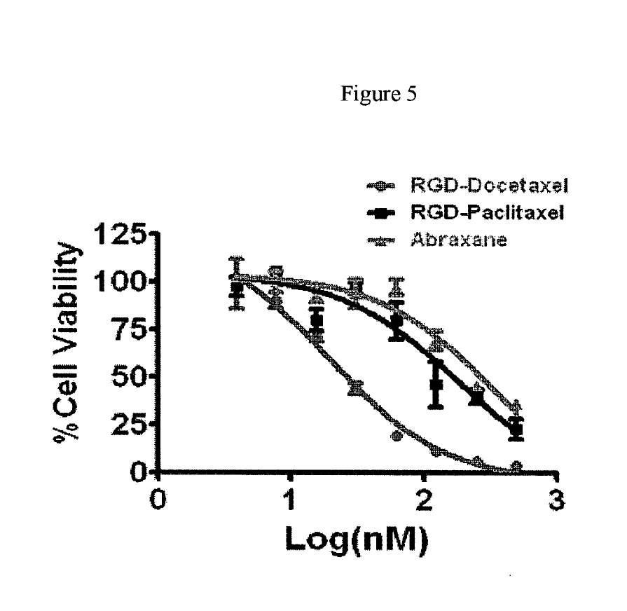

FIG. 5 graphically illustrates data from a cell viability assay comparing exemplary integrin αvβ3 targeted nanogel loaded with taxanes to ABRAXANE™ (albumin bound paclitaxel). The assays are done with M21 cells which express αvβ3. Nanogels are exposed to the cells at 4° C. for 10 min before being washed away. The cells are then returned to 37° C. for 72 h and cell viability is measured with XTT at 450 nm. EC50s are 20, 179, and 348 nM for RGD-docetaxel, RGD-paclitaxel, and ABRAXANE™, respectively. Error bars represent ±s.e.m.

In summary, the data in FIG. 5 demonstrates that these exemplary nanogels of the invention targeting integrin αvβ3 and carrying albumin bound docetaxel or paclitaxel increase their efficacy over Abraxane (untargeted albumin bound paclitaxel).

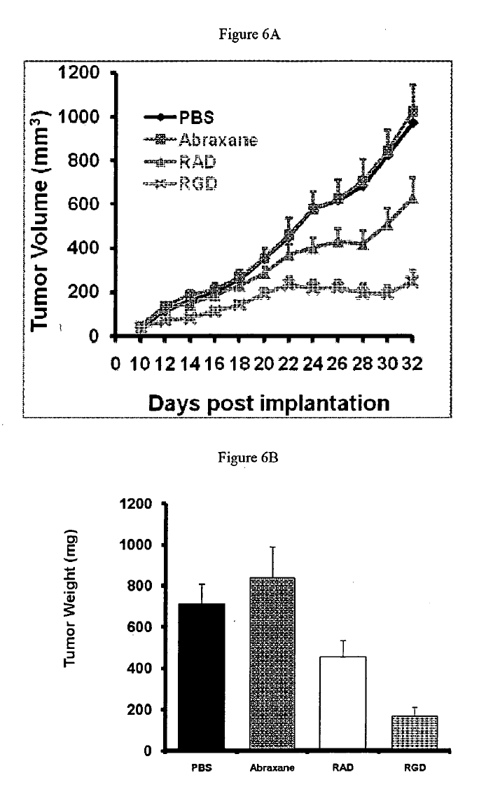

FIGS. 6A and 6B graphically illustrate data demonstrating that exemplary targeted nanogels of the invention are effective drug delivery vehicles for suppressing breast cancer. Orthotopic breast carcinoma model comparing the integrin αvβ3 targeted nanogel to ABRAXANE™. MDA-MB-231/LM-2 cells were injected into the mammary fat pad (#4) and allowed to establish a primary tumor. FIG. 6(A): Nanogels or ABRAXANE™ were iv injected qod from day 10 to day 28 at 1 mg/kg total drug. The nanogels (RGD or RAD) were loaded with docetaxel and compared to ABRAXANE™ for benchmarking. RAD is the control untargeted nanogel whereas RGD is the nanogel targeted to integrin αvβ3. RGD vs. ABRAXANE™ p=0.00005. FIG. 6(B): On day 32, mice were sacrificed and the breast tumors were resected and weighed.

In summary, the data in FIG. 6 demonstrates that the integrin αvβ3 targeted RGD-Docetaxel exemplary nanogel formulation of the invention suppresses orthotopic breast tumor growth. The untargeted RAD-docetaxel shows moderate activity when compared to the targeted formulation.

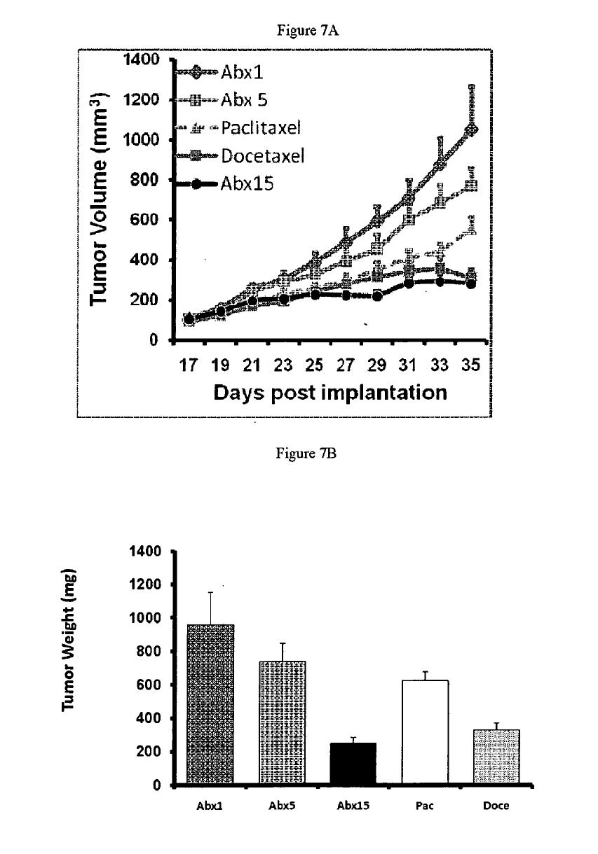

FIGS. 7A and 7B graphically illustrate data demonstrating that exemplary targeted nanogels of the invention as drug delivery vehicles require 15-fold less drug compared to ABRAXANE™. MDA-MB-231/LM-2 cells were injected into the mammary fat pad (#4) and allowed to establish a primary tumor. FIG. 7(A): Orthotopic breast carcinoma model comparing the integrin αvβ3 targeted nanogel to a dose response curve with Abraxane. Nanogels or ABRAXANE™ were iv injected qod from day 17 to day 33 at 1 mg/kg total drug for nanogels (paclitaxel or docetaxel) and at 1, 5, or 15 mg/kg for ABRAXANE™. RGD-docetaxel vs. ABRAXANE™ 1 mg/kg, p=0.003 and RGD-docetaxel vs. ABRAXANE™ 5 mg/kg, p=0.005. FIG. 7(B): On day 35, mice were sacrificed and the breast tumors were resected and weighed.

In summary, the data in FIG. 7 demonstrates that the integrin αvβ3 targeted RGD-Docetaxel nanogel formulations of the invention suppress orthotopic breast tumor growth at 1 mg/kg, whereas 15 mg/kg of paclitaxel in Abraxane is required for the same suppression.

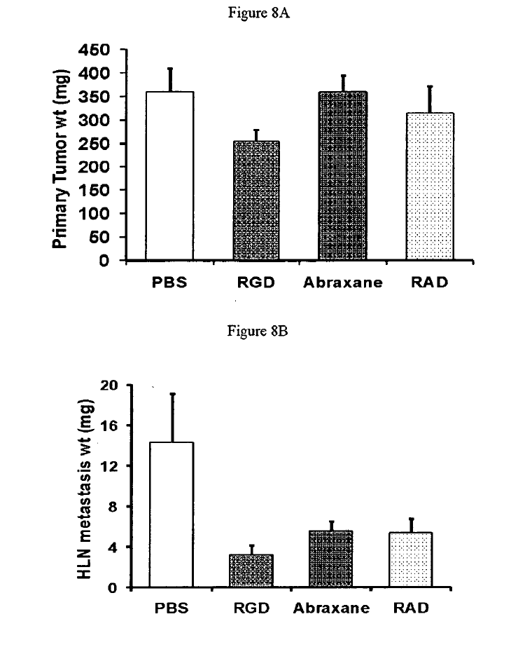

FIGS. 8A and 8B graphically illustrate data demonstrating that exemplary integrin αvβ3 targeted nanogels of the invention can effectively deliver docetaxel and suppress metastasis. Utilizing the R40P orthotopic pancreatic tumor model, we injected the docetaxel loaded nanogels (1 mg/kg qod, iv on day 5, 7, and 9). ABRAXANE™ was iv injected at 1 mg/kg total paclitaxel on the same qod schedule. FIG. 8(A): On day 11, the primary tumors were harvested and weighed. FIG. 8(B): Subsequently, the tumor burden found in the hepatic hilar lymph node was resected and weighed and denotes the metastatic burden. PBS vs. RGD-docetaxel, p=0.049.

In summary, the data in FIG. 8 demonstrates that the integrin αvβ3 targeted RGD-Docetaxel nanogel formulations of the invention suppress orthotopic pancreatic tumor growth and metastasis to the hepatic hilar lymph node. The untargeted RAD-docetaxel shows moderate activity when compared to the targeted formulation. ABRAXANE™ also shows some efficacy at suppressing metastatic lesions in the hepatic hilar lymph node.

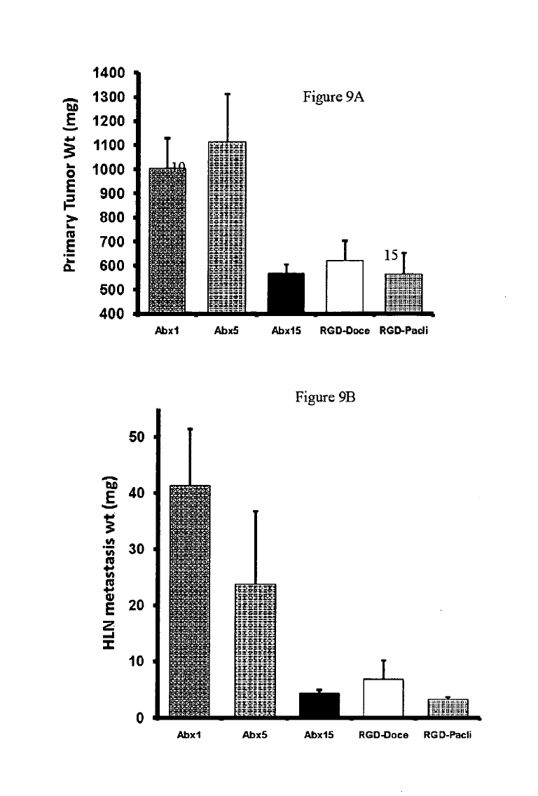

FIGS. 9A and 9B graphically illustrate data demonstrating that exemplary integrin αvβ3 targeted nanogels of the invention, RGD-Paclitaxel or RGD-Docetaxel, inhibit pancreatic primary tumor growth and metastasis at 15-fold lower close compared to ABRAXANE™. Utilizing the R40P orthotopic pancreatic tumor model, we injected the docetaxel loaded nanogels (1 mg/kg qod, iv on day 7, 9, 11, 13, 15, 17, and 19). ABRAXANE™ was intravenously (IV) injected at 1 mg/kg total paclitaxel on the same qod schedule. FIG. 9(A): On day 21, the primary tumors were harvested and weighed. For primary tumor Abx1 vs. ROD-paclitaxel, p=0.019. FIG. 9(B): Subsequently, the tumor burden found in the hepatic hilar lymph node was resected and weighed and denotes the metastatic burden. Abx1 vs. RGD-paclitaxel, p=0.005.

In summary, the data in FIG. 8 demonstrates that the integrin αvβ3 targeted RGD-Docetaxel or RGD-Paclitaxel nanogel formulations of the invention suppress orthotopic pancreatic tumor growth and metastasis at 1 mg/kg, whereas 15 mg/kg of paclitaxel in Abraxane is required for the same suppression. These targeted nanogel formulations of the invention provide a means to retain efficacy at low drug doses, which will likely reduce the side effects associated with the treatment.

A number of embodiments of the invention have been described. Nevertheless, it will be understood that various modifications may be made without departing from the spirit and scope of the invention. Accordingly, other embodiments are within the scope of the following claims.

TABLE 1
Summary of the EC50s from the cell viability
experiments in FIG. 2 and FIG. 3.

| Drug | Target | Class | EC50 (μM) |
|---|---|---|---|
| Docetaxel | β-tubulin | Taxane | 0.02 |
| Paclitaxel | β-tubulin | Taxane | 0.18 |
| Bortezomib | 26S-proteasome | Peptide mimetic | 0.04 |
| 17-AAG | Hsp90 | Antitumor antibiotic | 0.89 |
| Sorafenib | VEGFR2/RAF | Kinase inhibitor | 5.5 |
| Sunitinib | Broad RTK | Kinase inhibitor | 7.1 |
| Bosutinib | Src family | Kinase inhibitor | 5.4 |
| Dasatinib | Abl/Src family | Kinase inhibitor | 7.7 |

1. A product of manufacture or a composite nanostructure for in vitro, in vivo and/or ex vivo composition delivery comprising:
(a) (i) a polymer core, wherein the polymer comprises a plurality of monomers;
(ii) a first lipid or phospholipid layer or coating over (substantially covering) the polymer core;
(iii) a composition entirely contained within the polymer core, or composition substantially contained within the polymer core; and
(iv) a cell or tissue targeting composition covalently, ionically or non-covalently attached to the lipid or phospholipid such that a cell or tissue targeting of the cell or tissue targeting composition is positioned outward (to the exterior) of the product of manufacture or composite nanostructure; or
(b) the product of manufacture or a composite nanostructure of (a), wherein the nanoparticle formulation comprises cholesterol:DOPE:DSPC:DSPE-(PEO)₄-cRGDfK:DSPE-mPEG2000 at a 6:6:6:1:1 molar ratio.

2. The product of manufacture or composite nanostructure of claim 1, wherein the polymer core:
(a) is processed or manufactured to comprise a size in a range of between about 2, 3, 4, 5, 6, 7, 8, 9, 10, 11, 12, 13, 14, 15, 16, 17, 18, 19 or 20 or more to about 10, 50, 100, 200, 300, 400, 500, 600, 700, 800, 900 or 1000 or more nm; or, the product of manufacture or a composite nanostructure is processed or manufactured to have a size range of from between about 2, 3, 4, 5, 6, 7, 8, 9, 10, 11, 12, 13, 14, 15, 16, 17, 18, 19 or 20 or more to about 10, 50, 100, 200, 300, 400, 500, 600, 700, 800, 900 or 1000 or more nm;
(b) comprises or consists of one or more materials or compositions capable of absorbing hydrophobic molecules, hydrophilic molecules or hydrophobic molecules and hydrophilic molecules;
(c) comprises or consists of a material capable of forming a hydrogel, and/or the polymer core comprises or consists of random and/or block copolymers;
(d) comprises or consists of a polymer hydrogel core;
(e) comprises or consists of a methacrylic or acrylic ester monomer, an acrylamide (methacrylamide) monomer, an N-vinyl-2-pyrrolidone, a starch, ethylene glycol, hyaluran, chitose, and/or cellulose, or equivalents; or
(f) comprises or consists of a polypeptide, peptide or peptidomimetic, and optionally the polypeptide, peptide or peptidomimetic comprises or consists of a biopolymer, and optionally the biopolymer comprises or consists of an albumin or equivalent.

2-8. (canceled)

9. The product of manufacture or composite nanostructure of claim 1, wherein:
(a) the monomers, the polymer hydrogel and/or the polypeptide or peptidomimetic are cross-linked with a cross-linking agent; or the core comprises or consists of an unsaturated moiety capable of ultraviolet (uv) or thermal crosslinking to stabilize the core material
(b) the monomers, the polymer hydrogel and/or the polypeptide or peptidomimetic are cross-linked with a cross-linking agent or cross-linking mechanism comprising or consisting of a radical mechanism or a chemical crosslinking agent, and optionally the chemical crosslinking agent comprises or consists of a glutaraldehyde, or equivalent, and optionally the cross-linking agent comprises or consists of ethylene dimethacrylate, N,N-methylenediacrylamide, methylenebis(4-phenyl isocyanate), epichlarohydin glutaraldehyde, ethylene dimethacrylate, divinylbenzene and/or allyl methacrylate, or equivalent;
(c) the polymer core comprises or consists of a polylactide (PLA), a polyglycolide (PGA), a polylactide coglycolide (PLGA), a polycaprolactone (PCL), a copolymer of these materials, a polyanhydride, a polyortho ester, a biostable or bioinert hydrogel matrix-forming polymer, a water-containing gel, a polyvinylpyrrolidone (PVP), a polyethylene glycol (PEG), a polyethylene oxide (PEO), a polyacrylamide (PAA), polyvinyl alcohol (PVA), a biostable or bioinert matrix-forming polymer, a synthetic polymer, a methyl methacrylate, a butyl methacrylate and/or a dimethyl siloxane, or equivalent; or
(d) the cell or tissue targeting composition is covalently, ionically or non-covalently attached to the lipid or phospholipid via a cross-linking agent, and optionally the cross-linking agent comprises or consists of ethylene dimethacrylate, N,N-methylenediacrylamide, methylenebis(4-phenyl isocyanate), epichlarohydin glutaraldehyde, ethylene dimethacrylate, divinylbenzene and/or allyl methacrylate, or equivalent.

10-15. (canceled)

16. The product of manufacture or composite nanostructure of claim 1, further comprising a drug, a biological agent, a small molecule, a chemical, a cell, or an inorganic nanoparticle.

17. The product of manufacture or composite nanostructure of claim 1, wherein:
(a) the cell or tissue targeting composition comprises a ligand capable of specifically binding to an integrin, or equivalent, and optionally the integrin comprises or consists of (or is) avb3, a4b1 a5b1 and/or plaktin, or equivalent;
(b) the cell or tissue targeting composition comprises or consists of (or is) a linear peptide, a cyclic peptide or a synthetic organic molecule mimicking a peptide; or
(c) the cell or tissue targeting composition comprises or consists of (or is) an antibody.

18-20. (canceled)

21. The product of manufacture or composite nanostructure of claim 1, further comprising a second lipid layer or coating,
wherein optionally the second lipid layer or coating acts as a stealth component on a first lipid layer or coating monolayer to improve biological compatibility and increase in vivo circulation rates,
and optionally the first lipid layer or coating or the second lipid layer or coating further comprises a stabilizing molecule, and optionally the stabilizing molecule comprises or consists of (or is) a cholesterol or a colesteric analog, or comprises or consists of (or is) a crosslinkable lipid that is UV or heat sensitive.

22-24. (canceled)

25. The product of manufacture or composite nanostructure of claim 1, further comprising a component positioned (sandwiched) between the first and second lipid layer or coating to increase the melting range of the first and second lipid layer or coating monolayer component.

26. The product of manufacture or composite nanostructure of claim 1, further comprising a crosslinked lipid to stabilize the first and/or second lipid layer.

27. The product of manufacture or composite nanostructure of claim 1, wherein:
(a) the first and/or second lipid or phospholipid layer or coating comprises a free saturated or unsaturated fatty acid or an ester or an amide thereof; an anionic lipid, a cationic lipid, a zwitterionic lipid, a diacyl trialkylammonium propane and/or a quaternary ammonium compound, or equivalent
(b) the first and/or second lipid or phospholipid layer or coating comprises a mixture of oil-phase components, a squalane, a sterol, a ceramide, a neutral lipid or oil, a fatty acid, a lecithin, a phosphatidylcholine (PC) diacyl ester, a PC with saturated or unsaturated fatty acids or with aromatic acids such as dioleoyl-PC, dimyristoyl-PC (DMPC), dipalmitoyl-PC, distearoyl-PC (DSPC), diarachidonyl-PC (DAPC), and diphthaloyl-PC (DPPC); phosphatidylethanolamine (PE) diacyl esters with saturated or unsaturated fatty acids or with aromatic acids such as dioleoyl-PE, dimyristoyl-PE (DMPE), dipalmitoyl-PE (DPPE) and distearoyl-PE (DSPE); phosphatidylserine; phosphatidylglycerols, distearoylphosphatidylglycerol (DSPG); phosphatidylinositol; phosphatidic acids (PA), dipalmitoyl-PA (DPPA) and/or distearoyl-PA (DSPA), or equivalent; or
(c) the first and/or second lipid or phospholipid layer or coating comprises a lipid-bearing polymer such as chitin, hyaluronic acid, polyvinylpyrrolidone or polyethylene glycol (PEG), a pegylated DPPE (DPPE-PEG), phosphatidylcholine, phosphatidylethanolamine, sphingomyelin, phosphatidyl-glycerol, phosphatidylserine, phosphatidylinositols, phosphatidic acid, stearylamine, diacyloxy trimethylammonium propanes and/or diacyloxy dimethylammonium propane.

28-29. (canceled)

30. The product of manufacture or a composite nanostructure of claim 1, comprising for targeted delivery:
(a) a bioactive material, an anticancer cytostatic or cytotoxic agent, a gemcitabine (e.g., GEMZAR™) or a small molecule inhibitor of oncogenic or inflammatory proteins, a kinase inhibitor, a cytotoxic lipopeptide, a somocystinamide or a curacin A; or
(b) an antibiotic, an antidepressant, an anti-tumorigenic, an antiviral, a cytokine, a hormone, an imaging agent, a neurotransmitter, a nucleic acid, a stimulant, a regulating agent that turns genes and/or protein production on or off, a poly-functional alkylating agent, mechlorethamine, chlorambucil, melphalan, thiotepa, busulfan, cyclophosphamide, ifosfamide, an antimetabolite, methotrexate, 6-mercaptopumme, 6-thioguanme, 5-fluorouracil, 5-fluorodeoxyuridine, cytarabine, fludarabine, 2-chlorodeoxyadenosine, 2-deoxycoformycin, genicitabine, an antibiotic, doxorubicin, bleomycin, dactinomycin, daunorubicin, plicamycin, mitomycin C, mitoxantrone, a steroid, a hormonally active compound, androgen, fluoxymesterone, an anti-androgen, flutamide, an estrogen, ethinyl estradiol, diethylstilbestrol, an anti-estrogen, tamoxifen, a progestational agent, megestrol acetate, a luteinizing hormone-releasing hormone agonist, leuprolide, an aromatase inhibitor, aminoglutethimide, an adrenal cortical compound, dexamethasone, asparaginase, altretaimine, carmustine, lomustine, steptozotocin, mitotane, dacarbazine, hydroxyurea, etoposide, cisplatin, carboplatin, procarbazine, vinblastine, vincristine, levamisole, cis-retinoic acid, paclitaxel, docetaxel, a biologic agent, alpha-interferon, beta-interferon, interferon-gamma, tumor necrosis factor, erythropoietin, granulocyte colony-stimulating factor, granulocyte macrophage colony-stimulating factor, macrophage colony-stimulating factor or interleukin-1, interleukin-2 and combinations thereof.

31. (canceled)

32. A pharmaceutical composition or formulation for treating or ameliorating a disease or condition comprising:
(a) the product of manufacture or nanostructure of claim 1, wherein optionally the product of manufacture or nanostructure comprises or is contained within an implant or an intradermal or a subcutaneously transplantable assembly; or
(b) the pharmaceutical composition or formulation of (a), wherein the pharmaceutical composition or formulation is formulated for enteral or parenteral administration.

33-35. (canceled)

36. A device or nanodevice for treating or ameliorating a disease or condition comprising the product of manufacture or nanostructure of claim 1, wherein optionally the product of manufacture, nanostructure or nanodevice comprises or is contained within an implant or an intradermal or a subcutaneously transplantable assembly.

37. An implant comprising the product of manufacture or nanostructure of claim 1, wherein optionally the implant is an intradermal or a subcutaneously transplantable assembly.

38. An anti-nerve gas agent, or anti-toxin, device comprising the product of manufacture or nanostructure of claim 1, wherein optionally the product of manufacture or nanodevice comprises or is contained within an implant, or an intradermal or a subcutaneously transplantable assembly.
